(19) United States
(12) Patent Application Publication
(10) Pub. No.: US 2006/0182806 A1
(43) Pub. Date: Aug. 17, 2006

(54) Extended-release propranolol composition

(76) Inventors: Mintong Guo, Plainsboro, NJ (US); Suresh Palaniswamy, East Windsor, NJ (US); Ashish Anilbhai Patel, East Brunswick, NJ (US); Chad Michael Gassert, Bayonne, NJ (US); Pablo Davila, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/268,060

(22) Filed: Nov. 7, 2005

Related U.S. Application Data
(60) Provisional application No. 60/625,866, filed on Nov. 8, 2004.

(51) Int. Cl. A61K 9/26

(52) U.S. Cl. 424/470

(57) ABSTRACT

An extended-release pharmaceutical composition comprising a core and a coating, wherein said core comprises propranolol or a pharmaceutically acceptable salt thereof and at least one excipient, and said coating comprises at least one water-soluble polymer and at least one water-insoluble polymer, wherein said coating is heated at a temperature of about 30° C. to about 70° C. after being applied to said core.

### CONTINUITY INFORMATION

This application claims benefit of U.S. Provisional Application No. 60/625,866, filed Nov. 8, 2005, which in its entirety is herein incorporated by reference.

### FIELD OF THE INVENTION

The invention relates to a process for preparing an extended-release pharmaceutical composition comprising propranolol or a pharmaceutically acceptable salt thereof.

### BACKGROUND OF THE INVENTION

Propranolol hydrochloride (HCl) is chemically described as 1-(isopropylamino)-3-(1-naphthyloxy)-2-propranolol hydrochloride, and is a synthetic beta-(β)-adrennergic receptor-blocking agent, which is mainly used for the treatment of angina pectoris, cardiac arrhythmias and hypertension. Propranolol HCl specifically competes with β-adrenergic receptor-stimulating agents for available receptor sites. When access to Preceptor sites is blocked by propranolol HCl, the chronotropic, inotropic and vasodilator response to β-adrenergic stimulation are decreased proportionally. Due to the nature of these indications where propranolol HCl primarily targets, it offers much more benefit if propranolol HCl is delivered into the body through extended-release mechanism. Extended-release formulation can reduce the dosing frequency and improve the patients' compliance. In addition, extended-release can provide a stable drug concentration in the blood circulation system and avoid fluctuation of the blood pressure.

U.S. Pat. Nos. 3,337,628 and 3,520,919 describe propranolol, its acid-addition salts and processes of manufacture. GB Patent No. 995,800 describes pharmaceutical compositions containing propranolol.

U.S. Pat. No. 4,138,475 (the '475 patent) describes a sustained-release composition consisting of a hard gelatin capsule containing film coated spheroids. The spheroids contain, prior to coating, 40-65% by weight of propranolol or a pharmaceutically acceptable acid addition salt thereof, in admixture with non-water swellable microcrystalline cellulose, and the spheroids have a film coat comprising ethylcellulose or ethylcellulose and hydroxypropylmethyl cellulose. The spheroids are manufactured by extrusion and spheronization processes.

It would be desirable to prepare a pharmaceutical composition containing propranolol which is stable at a wide-range of temperature and RH conditions. In addition, the dissolution profile or release profile of such compositions should desirably be more consistent over time, as compared to prior art compositions.

### SUMMARY OF THE INVENTION

The invention provides an extended-release pharmaceutical composition comprising a core and a coating, wherein said core comprises propranolol or a pharmaceutically acceptable salt thereof and at least one excipient, and said coating comprises at least one water-soluble polymer and at least one water-insoluble polymer, wherein said coating is heated at a temperature of about 30° C. to about 70° C. after being applied to said core.

According to another aspect, the invention provides a process for preparing an extended-release pharmaceutical composition comprising:
(a) mixing propranolol or a pharmaceutically acceptable salt thereof and at least one excipient to form a premix;
(b) adding a solvent to the premix to form a wet granulation;
(c) extruding the wet granulation to form an extrudate;
(d) spheronizing the extrudate to form pellets;
(e) drying the pellets;
(f) coating the pellets, wherein said coating comprises at least one water-soluble polymer and at least one water-insoluble polymer; and
(g) heating the coated pellets at a temperature of about 30° C. to about 70° C. to form an extended-release pharmaceutical composition.

The pharmaceutical compositions of the invention have the following advantages:
(i) the composition provides steady drug plasma concentration during the intended therapeutic time, therefore reduce the dosing frequency;
(ii) the composition eliminates fluctuation of drug plasma concentration and reduce the side effect associated with peak drug concentration; and
(iii) the composition has a stable release profile over the time at different storing conditions.

### BRIEF DESCRIPTION OF THE DRAWINGS

FIG. 1 is a dissolution profile showing the release of propranolol HCl from coated pellets cured at 60° C. for 40 hours, without being stored, and after being stored for 2 and 4 weeks at 40° C. and 75% RH.

FIG. 2 is a dissolution profile showing the release of propranolol HCl from coated pellets cured at 40° C. for 24 hours, without being stored, and after being stored for 2 and 4 weeks at 40° C. and 75% RH.

### DETAILED DESCRIPTION OF THE INVENTION

The invention provides an extended-release pharmaceutical composition comprising a core and a coating, wherein said core comprises propranolol or a pharmaceutically acceptable salt thereof and at least one excipient, and said coating comprises at least one water-soluble polymer and at least one water-insoluble polymer, wherein said coating is heated at a temperature of about 30° C. to about 70° C., preferably about 40° C. to about 60° C., after being applied to said core. A preferred period of time for heating the coating is from about 5 hours to about 72 hours, more preferably from about 12 hours to about 48 hours. Most preferably, the coating is heated for a period of time from about 20 hours to about 40 hours. As used herein, “extended-release” means the propranolol or pharmaceutically acceptable salt thereof is released from the composition over a period of time, i.e., 24 hours, and includes controlled-release and sustained-release. As used herein, “polymer” includes copolymers, terpolymers, etc.

Preferably, the pharmaceutically acceptable salt of propranolol is an acid addition salt such as propranolol HCl.

The propranolol or pharmaceutically acceptable salt thereof is present in an amount of from about 10 weight percent (wt. %) to about 70 wt. %, based on the total weight of the core. Preferably, the propranolol or pharmaceutically acceptable salt thereof is present in an amount of from about 40 wt. % to about 65 wt. %, more preferably about 50 wt. % to about 60 wt. %, based on the total weight of the core. Typically, the amount of propranolol or pharmaceutically acceptable salt thereof in the compositions of the invention varies from about 50 mg to about 300 mg, preferably about 160 mg.

The water-insoluble polymer for use in the coatings of the invention is preferably ethyl cellulose. A preferred form of ethylcellulose is that having a viscosity in the range of 5-100 cps at 20° C. (U.S. National Formulary XIII) (content of ethoxy groups 44-51% by weight), and more preferably a viscosity of 10 cps at 20° C. (content of ethoxy groups 48-49% by weight).

The amount of the water-insoluble polymer in the coating compositions of the invention is from about 50 wt. % to about 99 wt. %, based on the weight of the coating. Preferably, the amount of the water-insoluble polymer is from about 80 wt. % to about 96 wt. %, more preferably about 85 wt. % to about 90 wt. %.

The water-soluble polymers used in the coating compositions of the invention have a viscosity of less than about 50 centipoise (cps), as determined using a Ubbelohde viscometer, 2% by weight of polymer in water, at a temperature of 20° C.±0.1° C., according to the American Society for Testing and Materials (ASTM, D-445). More preferably, the water-soluble polymers used in the coating compositions have a viscosity of about 1 cps to about 30 cps, most preferably, from about 5 cps to about 20 cps. It is within the scope of the invention for the water-soluble polymers to include at least one polymer having a viscosity of greater than 50 cps provided that such polymer is combined with at least one water-soluble polymer having a viscosity of less than 50 cps to yield a combination of polymers having a viscosity of from about 1 cps to about 50 cps.

The water-soluble polymer for use in the coatings of the invention is preferably selected from hydroxypropylmethyl cellulose, hydroxypropyl cellulose (HPC), hydroxyethyl cellulose, methyl cellulose, sodium carboxymethyl cellulose, hydroxyethylmethyl cellulose, ethylcarboxyethyl cellulose, polyvinylalcohol, sodium alginate, polyvinylpyrrolidone, vinyl acetate/crotonic acid copolymers, methyl methacrylic ester copolymers, maleic anhydride/methyl vinyl ether copolymers and poly(ethylene oxide). A mixture of water-soluble polymers may also be used. More preferably, the water-soluble polymer is selected from hydroxypropylmethyl cellulose, HPC and poly(ethylene oxide).

Hydroxypropylmethyl cellulose is a polymer which is available in many forms, including forms of different molecular weight, extremely different viscosity and different substitution grade. It is within the scope of the invention to use mixtures or blends of two or more different forms of hydroxypropylmethyl cellulose as the water-soluble polymer in the coating. A preferred form of hydroxypropyl methylcellulose is that having a viscosity in the range 3-100 cps at 20° C. (U.S. National Formulary XIII), and more preferably a viscosity of 15 cps at 20° C. For example, Methocel E15 LV Premium, which has a viscosity of 15 cps, and is available from Dow Chemical.

HPC is a partially substituted poly(hydroxypropyl) ether of cellulose. HPC is commercially-available in a number of different grades which have different solution viscosities. The molecular weight of the HPC ranges from about 50,000 to about 1,250,000. A preferred HPC is available from Aqualon under the trademark KLUCEL. Suitable grades of HPC include the following:
1) KLUCEL EF having a molecular weight of about 80,000;
2) KLUCEL LF having a molecular weight of about 95,000;
3) KLUCEL JF having a molecular weight of about 140,000;
4) KLUCEL GF having a molecular weight of about 370,000;
5) KLUCEL MF having a molecular weight of about 850,000; and
6) KLUCEL HF having a molecular weight of about 1,150,000.

Included within the term “HPC” is a low-substituted hydroxypropyl cellulose (L-HPC). The L-HPC useful in the compositions of the invention is available in a number of different grades which have different particle sizes and substitution levels, and which are classified on the basis of their percent hydroxypropoxy content. When dried at 105° C. for 1 hour, the L-HPC contains from about 5% to about 16% of hydroxypropoxy groups, preferably from about 10% to about 13% of hydroxypropoxy groups. Suitable grades of L-HPC include the following:
1) LH-11 having a hydroxypropoxy content of 11% and an average particle size of 50 microns;
2) LH-21 having a hydroxypropoxy content of 11% and an average particle size of 40 microns;
3) LH-31 having a hydroxypropoxy content of 11% and an average particle size of 25 microns;
4) LH-22 having a hydroxypropoxy content of 8% and an average particle size of 40 microns;
5) LH-32 having a hydroxypropoxy content of 8% and an average particle size of 25 microns;
6) LH-20 having a hydroxypropoxy content of 13%, and an average particle size of 40 microns; and
7) LH-30 having a hydroxypropoxy content of 13%, and an average particle size of 25 microns.

Preferred L-HPCs are commercially-available from Shin-Etsu Chemical Company under the trade designation L-HPC Grade LH-21 and LH-11.

The amount of the water-soluble polymer in the coating compositions of the invention is from about 1 wt. % to about 50 wt. %, based on the weight of the coating. Preferably, the amount of the water-soluble polymer is from about 5 wt. % to about 30 wt. %, more preferably about 70 wt. % to about 15 wt. %.

In addition to the water-insoluble polymer and water-soluble polymer, the coating may optionally contain a plasticizer. Examples of plasticizers include, but are not limited to, dibutyl sebacate; vegetable oil, e.g., castor oil or glycerol; or a glyceryl ester of a fatty acid, e.g., glyceryl triacetate or glyceryl monoricinoleate.

The extended-release pharmaceutical compositions of the invention may also contain one or more excipients that are generally employed in pharmaceutical formulations. Examples of such excipients are surfactants, diluents, binders, amino acids, solubilizers, disintegrants, fillers, lubricants, buffers, stabilizers, colorants, dyes, anti-oxidants, anti-adherents, preservatives and glidants. A combination of excipients may also be used. Such excipients are known to those skilled in the art, and thus, only a limited number will be specifically referenced.

Examples of fillers include microcrystalline cellulose, lactose, dibasic calcium phosphate dihydrate, calcium sulfate trihydrate and calcium sulfate dehydrate. A combination of fillers may also be used. A preferred filler for use in the core is microcrystalline cellulose.

Examples of lubricants include magnesium stearate, sodium stearate, calcium stearate, zinc stearate, talc, propylene glycol, PEG, stearic acid, vegetable oil, sodium benzoate, sodium lauryl sulfate, magnesium lauryl sulfate, mineral oil and polyoxyethylene monostearate. A combination of lubricants may also be used. A preferred lubricant is talc.

Examples of binders include gums, such as gum tragacanth, acacia gum and gelatin; microcrystalline cellulose, e.g., products known under the registered trademarks Avicel, Filtrak, Heweten or Pharmacel; hydroxyethyl cellulose and hydroxypropylmethyl cellulose; and polyvinyl pyrrolidone, e.g., Povidone.

Examples of glidants include silica, magnesium trisilicate, powdered cellulose, talc, calcium silicate and tribasic calcium phosphate. Colloidal silica, e.g., Aerosil, is particularly preferred.

Examples of disintegrants include:
(i) cross-linked polyvinylpyrrolidones, e.g., crospovidones, such as Polyplasdone® XL and Kollidon® CL;
(ii) alginic acid and sodium alginate;
(iii) methacrylic acid-divinylbenzene co-polymer salts, e.g., Amberlite® IRP-88; and
(iv) cross-linked sodium carboxymethylcellulose, available as, e.g., Ac-di-sol®, Primellose®, Pharmacel® XL, Explocel® and Nymcel® ZSX.
   Additional disintegrants also include hydroxypropylmethyl cellulose, croscarmellose sodium, polacrillin potassium, polyacrylates, such as Carbopol®, magnesium aluminium silicate and bentonite.

In a preferred embodiment of the invention, the core component of the compositions of the invention contain propranolol or a pharmaceutically acceptable salt thereof, and microcrystalline cellulose, and the coating comprises a mixture of ethylcellulose and hydroxypropylmethyl cellulose.

A preferred microcrystalline cellulose is, e.g., Avicel-PH-101 which has a nominal mean particle size of 50 μm, and is available from FMC Corporation. The microcrystalline cellulose is preferably present in an amount of from about 15 wt. % to about 70 wt. %, based on the weight of the core. Preferably, the microcrystalline cellulose is present in an amount of from about 25 wt. % to about 50 wt. %, more preferably about 30 wt. %, based on the weight of the core.

The pharmaceutical compositions of the invention can be prepared by any of the conventionally employed processing techniques, such as wet granulation. Examples of solvents to be used in the wet granulation process include water, methanol, ethanol, isopropanol, acetone and ethylene chloride. A combination of solvents may also be used. Preferably, the solvent is water.

In one embodiment of the invention, the composition of the invention is prepared by a process comprising:
(a) mixing propranolol or a pharmaceutically acceptable salt thereof and at least one excipient to form a premix;
(b) adding a solvent to the premix to form a wet granulation;
(c) extruding the wet granulation to form an extrudate;
(d) spheronizing the extrudate to form pellets;
(e) drying the pellets;
(f) coating the pellets, wherein said coating comprises at least one water-soluble polymer and at least one water-insoluble polymer; and
(g) heating the coated pellets at a temperature of about 30° C. to about 70° C. to form an extended-release pharmaceutical composition.

Drying techniques useful for drying the pellets include fluid bed, flash drying, ring drying, micron drying, tray drying, vacuum drying, radio-frequency drying and microwave drying.

The compositions of the invention are prepared in a solid dosage form suitable for oral administration. Solid dosage forms include capsules, caplets, pellets, powders and tablets. Preferably, the compositions are enclosed in a capsule, more preferably a hard gelatin capsule.

The following non-limiting examples illustrate further aspects of the invention.

### EXAMPLE 1

Preparation of Propranolol HCl 160 mg Capsules.

| Item No. | Ingredients | mg/capsule |
|---|---|---|
| 1 | Propranolol HCl | 160.0 |
| 2 | Microcrystalline Cellulose pH 101 | 40.0 |
| | (Avicel pH 101) | |
| 3 | Purified Water | q.s. |
| | Total | 200.0 |

The pellets are prepared by mixing 20 kg propranolol of hydrochloride and 8.52 kg of microcrystalline cellulose in a 150 L Fielder Vertical High Shear mixer for 5 minutes to form a premix. Deionized water, 10 kg, is slowly added to the premix in the mixer and mixing is continued for an additional 3 minutes after addition of the water is completed to form wet granules. The wet granulation is discharged from the mixer and extruded through a Niro Nica E-140 Extruder with an impeller speed at 40 rpm and a feeder speed at 20 rpm to give cylindrical extrudate of nominally 1 mm diameter. The extrudate is place in a Niro Nica S-450 Spheroniser in which the rotating disc rotated at 600 rpm for less than 5 minutes. The resulting pellets are placed on trays and dried in an oven set at about 50° C. for about 12 hours wherein the Loss on Drying (LOD) of the pellets is no more than 1.5%. The dried pellets are screened using a Sweco mechanical screen with two screens #14 and #20. The pellets with a particle size between #14 and #20 screens are collected (0.5-2 mm). The over-and under-sized pellets are discarded.

### EXAMPLE 2

Preparation of Propranolol HCl 160 mg Capsules.

| Item No. | Ingredients | mg/capsule |
|---|---|---|
| 1 | Propranolol HCl | 160.0 |
| 2 | Microcrystalline Cellulose | 68.6 |
| | (Avicel pH 101) | |
| 3 | Purified Water | q.s. |
| | Total | 228.6 |

The capsules are prepared according to the procedure set forth in Example 1.

### EXAMPLE 3

Preparation of Propranolol HCl 160 mg Capsules.

| Item No. | Ingredients | mg/capsule |
|---|---|---|
| 1 | Propranolol HCl | 160.0 |
| 2 | Microcrystalline Cellulose | 100.7 |
| | (Avicel pH 101) | |
| 3 | Purified Water | q.s. |
| | Total | 260.7 |

The capsules are prepared according to the procedure set forth in Example 1.

### EXAMPLE 4

Preparation of Propranolol HCl 160 mg Capsules.

| Item No. | Ingredients | mg/capsule |
|---|---|---|
| 1 | Propranolol HCl | 160.0 |
| 2 | Microcrystalline Cellulose | 160.0 |
| | (Avicel pH 101) | |
| 3 | Purified Water | q.s. |
| | Total | 320.0 |

The capsules are prepared according to the procedure set forth in Example 1.

### EXAMPLE 5

Coating Composition.

| | Composition | Amount per |
|---|---|---|
| Ingredient | (%) | gram (mg) |
| Propranolol HCl Pellets (Example 2) | 93.15 | 931.45 |
| Ethylcellulose, NF (Ethocel Standard | 2.93 | 29.34 |
| 10FP Premium) | | |
| Hypromellose, USP (Methocel E15LV) | 0.33 | 3.26 |
| Dibutyl Sebacate, NF | 0.65 | 6.52 |
| Micro Talc, USP | 2.45 | 24.45 |
| Silicon Dioxide, USP/NF (Syloid 244FP) | 0.50 | 4.98 |
| Isopropyl Alcohol, USP | * | Removed |
| Purified Water, USP | * | Removed |
| Total Weight | 100.01 | 1000.00 |

The coating composition is prepared as follows: 18.5 kg of isopropyl alcohol is placed into a 50 L container and 0.66 kg of Ethocel 10FP is added with mixing until a clear solution is obtained. Methocel E15LV, 0.075 kg, is added with mixing. Dibutyl sebacate, 0.15 kg, is added with mixing for approximately 0.5 hour. Micro talc, 0.55 kg, is added with mixing for approximately 0.5 hour.

The pellets prepared in Example 1 were coated with the coating composition set forth above in this example using a Glatt GPCG-5 fluid bed with 12″ Wurster insert. The temperature of the coated pellets is maintained about 35° C. The coated pellets are screened using a Sweco mechanical screen with two screens #14 and #20. The coated pellets with a particle size between #14 and #20 screens are collected (0.5-2 mm).

### EXAMPLE 6

The coated pellets prepared in Example 5 are heated in an oven set at 60° C. for 40 hours. The coated pellets which are heated are evaluated in a dissolution study. A USP Type I dissolution apparatus is used which is set at 100 rpm, 320 nm UV, first phase pH 1.2 buffer solution, and second phase pH 6.8 buffer solution.

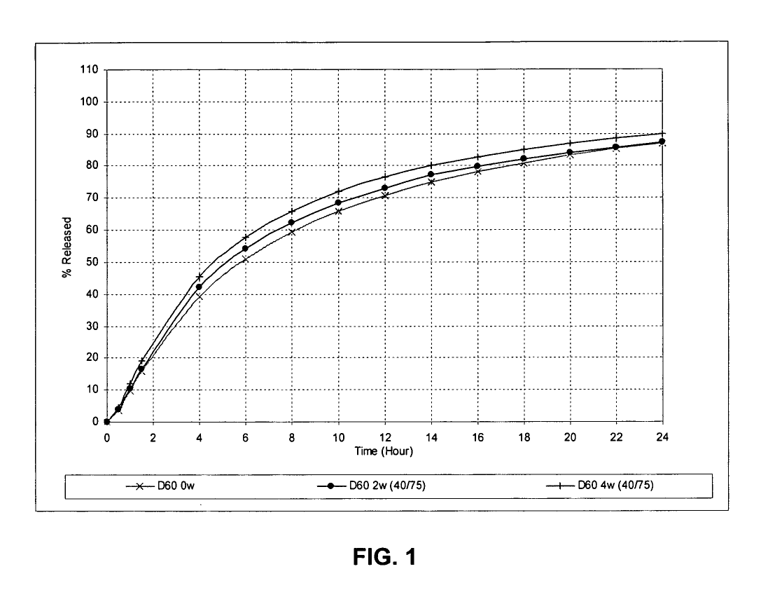

Three samples of the coated pellets which are subjected to heating after being coated are evaluated over a period of 24 hours: an initial sample, a sample after being stored at 40° C. and 75% RH for 2 weeks, and a sample after being stored at 40° C. and 75% RH for 4 weeks. The test results are summarized in FIG. 1.

FIG. 1 shows that the release of propranolol HCl from the coated pellets which are heated at 60° C. for 40 hours is constant over a period of 24 hours. In addition, FIG. 1 shows that the drug release profile of the pellets remain stable even after being stored for 4 weeks at 40° C. and 75% RH.

### EXAMPLE 7

The coated pellets prepared in Example 5 are heated in an oven set at 60° C. for 40 hours. The coated pellets which are heated are evaluated in a dissolution study. A USP Type I dissolution apparatus is used which is set at 100 rpm, 320 nm UV, first phase pH 1.2 buffer solution, and second phase pH 6.8 buffer solution.

Three samples of the coated pellets which are heated are evaluated over a period of 24 hours: an initial sample, a sample after being stored at 30° C. and 60% RH for 2 weeks, and a sample after being stored at 30° C. and 60% RH for 4 weeks. The test results are summarized in Table 1.

TABLE 1

| | | 2 Weeks at | 4 Weeks at |
|---|---|---|---|
| Time (hour) | Initial | 30° C. and 60% RH | 30° C. and 60% RH |
| 0 | 0 | 0 | 0 |
| 0.5 | 3.7 | 3.9 | 4.9 |
| 1 | 9.8 | 9.5 | 11.6 |
| 1.5 | 15.8 | 14.8 | 18.1 |
| 4 | 39.3 | 38.8 | 41.9 |
| 6 | 51.1 | 50.4 | 54.1 |
| 8 | 59.5 | 58.9 | 62.1 |
| 10 | 65.9 | 65.4 | 68.0 |
| 12 | 70.8 | 70.5 | 72.6 |
| 14 | 74.8 | 74.5 | 76.2 |
| 16 | 78.1 | 77.7 | 79.1 |
| 18 | 80.9 | 80.3 | 81.6 |
| 20 | 83.3 | 82.7 | 83.7 |
| 22 | 85.3 | 84.6 | 85.6 |
| 24 | 87.1 | 86.2 | 87.2 |

The results in Table 1 show that the release of propranolol HCl from the coated pellets which are heated at 60° C. for 40 hours is constant over a period of 24 hours. In addition, Table 1 shows that the drug release profile of the pellets remain stable even after being stored for 4 weeks at 30° C. and 60% RH.

### EXAMPLE 8

The coated pellets prepared in Example 5 are heated in an oven set at 50° C. for 24 hours. The coated pellets which are heated are evaluated in a dissolution study. A USP Type I dissolution apparatus is used which is set at 100 rpm, 320 nm UV, first phase pH 1.2 buffer solution, and second phase pH 6.8 buffer solution.

Three samples of the coated pellets which are heated are evaluated over a period of 24 hours: an initial sample, a sample after being stored at 25° C. and 60% RH for 2 weeks, and a sample after being stored at 25° C. and 60% RH for 4 weeks. The test results are summarized in Table 2.

TABLE 2

| | | 2 Weeks at | 4 Weeks at |
|---|---|---|---|
| Time (hour) | Initial | 25° C. and 60% RH | 25° C. and 60% RH |
| 0 | 0 | 0 | 0 |
| 0.5 | 0.9 | 1.2 | 1.0 |
| 1 | 2.5 | 3.4 | 3.0 |
| 1.5 | 4.8 | 5.9 | 5.6 |
| 4 | 18.1 | 20.0 | 19.5 |
| 6 | 27.2 | 30.2 | 28.5 |
| 8 | 34.8 | 38.2 | 36.1 |
| 10 | 41.2 | 44.7 | 42.4 |
| 12 | 46.6 | 50.2 | 47.9 |
| 14 | 51.4 | 54.9 | 52.6 |
| 16 | 55.6 | 58.9 | 56.6 |
| 18 | 59.4 | 62.3 | 60.2 |
| 20 | 62.6 | 65.4 | 63.6 |
| 22 | 65.6 | 67.9 | 66.6 |
| 24 | 68.2 | 70.6 | 69.2 |

The results in Table 2 show that the release of propranolol HCl from the coated pellets which are heated at 50° C. for 24 hours remain essentially identical during storage conditions of 40° C. and 75% RH for 4 weeks.

### EXAMPLE 9

The coated pellets prepared in Example 5 are heated in an oven set at 50° C. for 24 hours. The coated pellets which are heated are evaluated in a dissolution study. A USP Type I dissolution apparatus is used which is set at 100 rpm, 320 nm UV, first phase pH 1.2 buffer solution, and second phase pH 6.8 buffer solution.

Three samples of the coated pellets which are heated are evaluated over a period of 24 hours: an initial sample, a sample after being stored at 30° C. and 60% RH for 2 weeks, and a sample after being stored at 30° C. and 60% RH for 4 weeks. The test results are summarized in Table 3.

TABLE 3

| | | 2 Weeks at | 4 Weeks at |
|---|---|---|---|
| Time (hour) | Initial | 30° C. and 60% RH | 30° C. and 60% RH |
| 0 | 0 | 0 | 0 |
| 0.5 | 0.9 | 1.4 | 1.8 |
| 1 | 2.5 | 3.8 | 5.5 |
| 1.5 | 4.8 | 6.5 | 9.4 |
| 4 | 18.1 | 21.5 | 27.1 |
| 6 | 27.2 | 31.7 | 37.2 |
| 8 | 34.8 | 39.7 | 45.0 |
| 10 | 41.2 | 46.1 | 51.1 |
| 12 | 46.6 | 51.6 | 56.2 |
| 14 | 51.4 | 56.1 | 60.5 |
| 16 | 55.6 | 60.2 | 64.3 |
| 18 | 59.4 | 63.7 | 67.7 |
| 20 | 62.6 | 66.8 | 70.6 |
| 22 | 65.6 | 69.5 | 73.3 |
| 24 | 68.2 | 71.9 | 75.7 |

The results in Table 3 show that the release of propranolol HCl from the coated pellets which are heated at 50° C. for 24 hours is constant over a period of 24 hours. In addition, Table 3 shows that the pellets remain stable even after being stored for 4 weeks at 30° C. and 60% RH.

### EXAMPLE 10

The coated pellets prepared in Example 5 are heated in an oven set at 40° C. for 24 hours. The coated pellets which are heated are evaluated in a dissolution study. A USP Type I dissolution apparatus is used which is set at 100 rpm, 320 nm UV, first phase pH 1.2 buffer solution and second phase pH 6.8 buffer solution.

Three samples of the coated pellets which are heated are evaluated over a period of 24 hours: an initial sample, a sample after being stored at 30° C. and 60% RH for 2 weeks, and a sample after being stored at 30° C. and 60% RH for 4 weeks. The test results are summarized in Table 4.

TABLE 4

| | | 2 Weeks at | 4 Weeks at |
|---|---|---|---|
| Time (hour) | Initial | 30° C. and 60% RH | 30° C. and 60% RH |
| 0 | 0 | 0 | 0 |
| 0.5 | 0.8 | 1.5 | 1.0 |
| 1 | 2.7 | 4.1 | 3.0 |
| 1.5 | 5.0 | 7.1 | 5.5 |
| 4 | 17.5 | 21.9 | 18.5 |
| 6 | 26.2 | 32.1 | 27.3 |
| 8 | 33.5 | 40.0 | 34.6 |
| 10 | 39.7 | 46.4 | 40.8 |
| 12 | 45.2 | 51.7 | 46.1 |
| 14 | 49.9 | 56.3 | 50.7 |
| 16 | 54.1 | 60.2 | 54.8 |
| 18 | 57.8 | 63.6 | 58.4 |
| 20 | 61.1 | 66.7 | 61.7 |
| 22 | 64.1 | 69.4 | 64.6 |
| 24 | 66.9 | 71.8 | 67.3 |

The results in Table 4 show that the release of propranolol HCl from the coated pellets which are heated at 40 CC for 24 hours is stable at the storage conditions of 30° C. and 60% RH, and the drug release profile or dissolution remains essentially the same over time.

### EXAMPLE 11 (COMPARATIVE)

No Heating Step Applied to Coated Pellets.

The coated pellets prepared in Example 5 are evaluated in a dissolution study without subjecting the coated pellets to a heating step. A USP Type I dissolution apparatus is used which is set at 100 rpm, 320 nm UV, first phase pH 1.2 buffer solution and second phase pH 6.8 buffer solution.

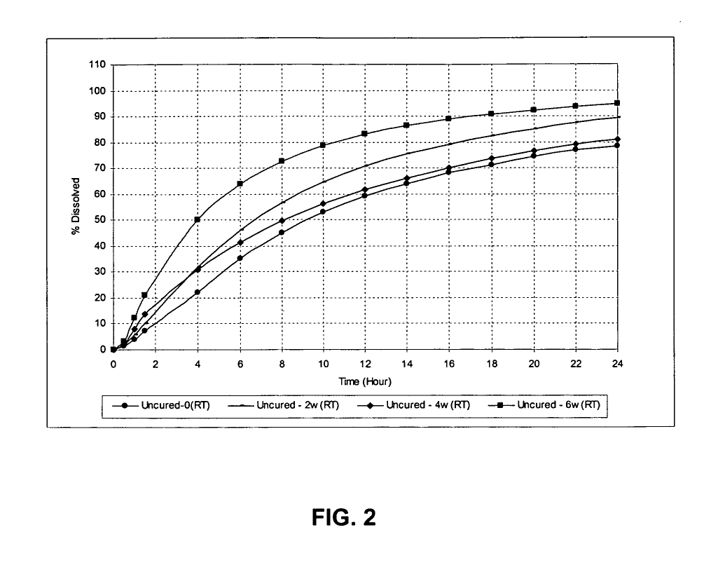

Three samples of the coated pellets are evaluated: an initial sample, a sample after being stored at 40° C. and 75% RH for 2 weeks, and a sample after being stored at 40° C. and 75% RH for 4 weeks. The test results are summarized in FIG. 2.

FIG. 2 shows that the release of propranolol HCl from the coated pellets is not constant at the storage conditions of 40° C. and 75% RH. In addition, FIG. 2 shows that the drug release from the pellets increases with the storage time.

While the invention has been described with particular reference to certain embodiments thereof, it will be understood that changes and modifications may be made by those of ordinary skill within the scope and spirit of the following claims:

What is claimed is:

1. An extended-release pharmaceutical composition comprising a core and a coating, wherein said core comprises propranolol or a pharmaceutically acceptable salt thereof and at least one excipient, and said coating comprises at least one water-soluble polymer and at least one water-insoluble polymer, wherein said coating is heated at a temperature of about 30° C. to about 70° C. after being applied to said core.

2. The composition according to claim 1, wherein the pharmaceutically acceptable salt of propranolol is propranolol hydrochloride.

3. The composition according to claim 1, wherein the propranolol or pharmaceutically acceptable salt thereof is present in an amount of from about 10 weight perfent (wt. %) to about 70 wt. %, based on the total weight of the composition.

4. The composition according to claim 3, wherein the propranolol or pharmaceutically acceptable salt thereof is present in an amount of from about 50 wt. % to about 60 wt. %.

5. The composition according to claim 1, wherein the excipient in the core is microcrystalline cellulose.

6. The composition according to claim 5, wherein the microcrystalline cellulose is present in an amount of from about 15 wt. % to about 70 wt. %, based on the weight of the core.

7. The composition according to claim 6, wherein the microcrystalline cellulose is present in an amount of from about 25 wt. % to about 50 wt. %.

8. The composition according to claim 1, wherein the water-insoluble polymer is ethyl cellulose.

9. The composition according to claim 8, wherein the ethylcellulose has a viscosity of 10 centipoise (cps) at 20° C.

10. The composition according to claim 1, wherein the water-insoluble polymer is present in an amount of from about 50 wt. % to about 99 wt. %, based on the weight of the coating.

11. The composition according to claim 10, wherein the water-insoluble polymer is present in an amount of from about 85 wt. % to about 90 wt. %, based on the weight of the coating.

12. The composition according to claim 1, wherein the water-soluble polymer is selected from the group consisting of hydroxypropylmethyl cellulose, hydroxypropyl cellulose (HPC), hydroxyethyl cellulose, methyl cellulose, sodium carboxymethyl cellulose, hydroxyethylmethyl cellulose, ethylcarboxyethyl cellulose, polyvinylalcohol, sodium alginate, polyvinylpyrrolidone, vinyl acetate/crotonic acid copolymers, methyl methacrylic ester copolymers, maleic anhydride/methyl vinyl ether copolymers, poly(ethylene oxide) and mixtures thereof.

13. The composition according to claim 12, wherein the water-soluble polymer is selected from the group consisting of hydroxypropylmethyl cellulose, HPC and poly(ethylene oxide).

14. The composition according to claim 13, wherein the water-soluble polymer is hydroxypropylmethyl cellulose.

15. The composition according to claim 14, wherein the hydroxypropyl methylcellulose has a viscosity of 15 cps at 20° C.

16. The composition according to claim 1, wherein the water-soluble polymer is present in an amount of from about 1 wt. % to about 50 wt. %, based on the weight of the coating.

17. The composition according to claim 16, wherein the water-soluble polymer is present in an amount of from about 7 wt. % to about 15 wt. %, based on the weight of the coating.

18. The composition according to claim 1, wherein the coating additionally comprises a plasticizer.

19. The composition according to claim 1, wherein the coating is heated at a temperature of about 40° C. to about 60° C.

20. The composition according to claim 1, wherein the coating is heated for a period of from about 5 to about 72 hours.

21. The composition according to claim 20, wherein the coating is heated for a period of from about 20 to about 40 hours.

22. The composition according to claim 1, wherein the composition is in a form selected from the group consisting of a capsule, caplet, powder and tablet.

23. The composition according to claim 22, in the form of a hard gelatin capsule.

24. A process for preparing an extended-release pharmaceutical composition comprising:
(a) mixing propranolol or a pharmaceutically acceptable salt thereof and at least one excipient to form a premix;
(b) adding a solvent to the premix to form a wet granulation;
(c) extruding the wet granulation to form an extrudate;
(d) spheronizing the extrudate to form pellets;
(e) drying the pellets;
(f) coating the pellets, wherein said coating comprises at least one water-soluble polymer and at least one water-insoluble polymer; and
(g) heating the coated pellets at a temperature of about 30° C. to about 70° C. to form an extended-release pharmaceutical composition.
